(19) United States
(12) Patent Application Publication
(10) Pub. No.: US 2010/0136684 A1
(43) Pub. Date: Jun. 3, 2010

(54) Natural IgM Antibodies

(76) Inventors: Michael C. Carroll, Wellesley, MA (US); Francis D. Moore, JR., Medfield, MA (US); Herbert B. Hechtman, Chestnut Hill, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/550,065

(22) Filed: Aug. 28, 2009

Related U.S. Application Data
(63) Continuation-in-part of application No. 12/259,767, filed on Oct. 28, 2008.
Application No. 12/259,767 is a continuation of application No. 11/069,834, filed on Mar. 1, 2005, now Pat. No. 7,442,783.
(60) Provisional application No. 60/549,123, filed on Mar. 1, 2004.
(60) Provisional application No. 60/588,648, filed on Jul. 16, 2004.

(51) Int. Cl. C07K 16/00; C07K 14/00; C07K 7/08; C07K 7/06; C12N 15/11; C12N 15/13; C12N 15/85; C12N 5/10

(52) U.S. Cl. 435/331; 530/387.9; 530/350; 530/327; 530/329; 536/23.1; 536/23.53; 435/320.1

(57) ABSTRACT

The invention provides natural IgM antibodies that may be used to induce inflammatory diseases or disorders such as ischemia-reperfusion injury to create animal models of such inflammatory diseases or disorders.

### 1. CROSS-REFERENCE TO RELATED APPLICATIONS

This application is a continuation-in-part application of U.S. application Ser. No. 12/259,767, filed Oct. 28, 2008, now pending, which is a continuation of U.S. application Ser. No. 11/069,834, now U.S. Pat. No. 7,442,783, which claims the benefit of U.S. Provisional Application No. 60/588,648, filed on Jul. 16, 2004 and U.S. Provisional Application No. 60/549,123 filed on Mar. 1, 2004; the contents of each of these applications are specifically incorporated by reference herein.

### 2. GOVERNMENT SUPPORT

This invention was made with government support under grant No. GM52585, GM24891, and GM07560 from the National Institutes of Health. The government has certain rights in the invention.

### 3. BACKGROUND OF THE INVENTION

Nucleated cells are highly sensitive to hypoxia and even short periods of ischemia in multi-cellular organisms can have dramatic effects on cellular morphology, gene transcription, and enzymatic processes. Mitochondria, as the major site of oxygen metabolism, are particularly sensitive to changes in oxygen levels and during hypoxia release reactive oxygen species that chemically modify intracellular constituents such as lipids and proteins. Clinically these effects manifest as an inflammatory response in the patient. Despite intensive investigations of cellular responses to hypoxia little is known regarding the initiation of acute inflammation.

Acute inflammatory responses can result from a wide range of diseases and naturally occurring events such as stroke and myocardial infarction. Common medical procedures can also lead to localized and systemic inflammation. Left untreated inflammation can result in significant tissue loss and may ultimately lead to multi-system failure and death. Interfering with the inflammatory response after injury may be one method to reduce tissue loss.

Inflammatory diseases and acute inflammatory responses resulting from tissue injury, however, cannot be explained by cellular events alone. Accumulating evidence supports a major role for the serum innate response or complement system in inflammation. Studies to date have looked at tissue injury resulting from ischemia and reperfusion as one type of inflammatory disorder that is complement dependent. For example, in the rat myocardial model of reperfusion injury, pretreatment of the rats with the soluble form of the complement type 1 receptor dramatically reduced injury. Understanding how complement activation contributes to an inflammatory response is an area of active investigation.

Inflammatory diseases or disorders are potentially life-threatening, costly, and affect a large number of people every year. New research tools for studying inflammatory diseases or disorders would aid the identification of new therapeutics for treating such diseases or disorders. Thus, effective research tools for studying inflammatory diseases or disorders are needed.

### 4. SUMMARY OF THE INVENTION

In one aspect, the invention features isolated natural immunoglobulins, in particular natural IgMs, that bind to ischemic antigen. The natural IgM antibodies are capable of activating complement, thereby inducing an immune response to ischemic antigen. In one embodiment, the antibody is produced by ATCC Accession Number PTA-3507. In another embodiment, the antibody has a light chain variable region comprising the amino acid sequence shown as SEQ ID NO: 8. In yet another embodiment, the antibody has a heavy chain variable region comprising the amino acid sequence shown as SEQ ID NO: 2.

In a further aspect, the invention features methods of inducing an immune response to an ischemic antigen in a non-human animal by administering to the animal a natural antibody described herein. Administration of the antibody to the non-human animal mimics inflammatory diseases or disorders in the animal such as ischemia-reperfusion injury, thereby creating animal models of such inflammatory diseases or disorders.

Other features and advantages of the invention will be apparent based on the following Detailed Description and Claims.

### 5. BRIEF DESCRIPTION OF THE DRAWINGS

FIG. 1 shows an IgM heavy chain sequence of B-1 hybridoma 22A5. (A) shows the IgM^{CM-22} (or 22A5 IgM) heavy chain nucleic acid sequence (SEQ ID NO: 1) and (B) shows the amino acid sequence corresponding to the heavy chain sequence of SEQ ID NO: 1 (SEQ ID NO: 2). Framework regions (FVWR) and complementarity-determining regions (CDR) are indicated above the nucleotides.

FIG. 2 shows an IgM light chain sequence of B-1 hybridoma 22A5. The IgM^{CM-22} (or 22A5 IgM) light chain nucleic acid sequence is shown (SEQ ID NO: 7). The amino acid sequence corresponding to the light chain sequence of SEQ ID NO: 7 is shown (SEQ ID NO: 8). Framework-regions (FVWR) and complementarity-determining regions (CDR) are indicated above the amino acids.

FIG. 3 is a bar graph depicting changes in intestinal permeability of inbred mice after intestinal ischemia and reperfusion or no injury (sham). WT represents parent strain for Cr2−/− mice. Cr2−/− was reconstituted with pooled IgG or IgM or saline control. Pooled IgM or IgG (0.5 mg) was administered intravenously approximately 1 hour before treatment. Values are means+standard error; n equals the number of mice in experimental groups.

FIG. 4 demonstrates reconstitution of I/R injury in antibody deficient mice (RAG-1) by pooled IgM from a single B-1 cell hybridoma clone. IgM or saline was injected intravenously 30 minutes before initial laparotomy. At the end of reperfusion, blood is obtained and permeability index is calculated as the ratio of ¹²⁵I counts of dried intestine versus that of blood. Values represent means±standard error; n equals the numbers of mice used in experimental groups. 132 WT plus normal saline; 2=RAG plus normal saline; 3=RAG plus IgM hybridoma CM-22; 4=WT sham control.

FIG. 5 is a schematic diagram of the proposed role for complement and complement receptors in positive selection of peritoneal B-1 lymphocytes.

FIG. 6A is an immunoblot showing the immune precipitation of reperfusion injury (RI) specific antigens. Detection of a unique band (arrow) at approximately 250 kDa on a SDS-PAGE (10%). Size markers are indicated on the left. Intestinal lysates were prepared from RAG-1^{-/-} mice reconstituted with IgM^{CM-22} and either sham control (no ischemia) or subjected to ischemia followed by reperfusion for 0 or 15 min.

FIG. 6B is a series of graphs showing results of in vitro binding assays of IgM^{CM-22} to the isoforms of non-muscle myosin heavy chain-II (NMHC-II). ELISA plates were coated with monoclonal antibodies for 3 different isoforms of NMHC-II (upper left: isoform A, upper right: isoform B, lower left: isoform C and lower right: anti-pan myosin antibody). Bound myosin heavy chain from intestinal lysates was detected by IgM^{CM-22} or IgM^{CM-31}. The results represent mean±standard error of OD 405 nm units and are representative of triplicate samples.

FIG. 6C is a photomicrograph and a scatter plot showing the restoration of RI injury by anti-pan myosin antibody in RAG-1^{-/-} mice. RAG-1^{-/-} mice were reconstituted with affinity purified anti-pan myosin followed by RI surgery. The left panels represents morphologies of RAG-1^{-/-} animals with saline control and with anti-pan myosin treatment. The right panel is the pathology scores of intestinal injury. The scatter plot (right panel) represents the pathology scores where each symbol represents a single animal.

FIG. 7A is a series of photomicrographs showing that the N2 self-peptide blocking RI in RAG-1^{-/-} mice. Two upper panels show representative sections prepared following RI treatment in RAG-1^{-/-} mice with IgM^{CM-22} alone or mixed with N2 self-peptide. Two lower panels are representative sections prepared from WT mice treated for intestinal RI, which received either saline or N2 peptide 5 minutes prior to reperfusion.

FIG. 7B is a scatter plot indicating mean pathology score of each group of animals treated as in panel A. Two columns on left represent RAG-1−/− mice reconstituted with IgM CM22 with or with out N2 peptide, respectively. The two columns on the right represent WT mice with or without N2 peptide, respectively, prior to treatment in the IR intestinal model. Each symbol represents one animal. Asterisk indicates significant protection, P<0.05 as determined by Student's t test of the N2 treated vs untreated groups.

FIG. 7C is a series of photomicrographs showing the prevention of the activation of classical pathway of complement in intestinal RI by the self-peptide N2. Representative cryosections of intestinal tissues were harvested following intestinal RI and treated with an antibody specific for the mouse IgM, C4 or C3 (400× magnification). IgM^{CM-22}-reconstituted RAG-1^{-/-} mice without pretreatment with the self-peptide N2 are in panels i-iv or with the self-peptide N2 are in panels v-viii. Wild type mice without pretreatment with the self-peptide N2 are in panels ix-xii or with pretreatment with the self-peptide N2 are in panels xiii-xvi. The tissue in panels i, iii, v, vii, ix, xiii, xi, xv were stained with anti-IgM-biotin followed by Streptavidin-Alexa-568 (red) and counterstained with DAPI (violet). Panels ii, vi, x, and xiv were stained with anti-C4-FITC (green). Panels iv, viii, xii, xvi were stained with anti-C3-FITC (green).

### 6. DETAILED DESCRIPTION

### 6.1. Definitions

For convenience, certain terms employed in the specification, examples, and appended claims are provided. Unless defined otherwise, all technical and scientific terms used herein have the same meaning as commonly understood by one of ordinary skill in the art to which this invention belongs.

“A” and “an” are used herein to refer to one or to more than one (i.e., to at least one) of the grammatical object of the article. By way of example, “an element” means one element or more than one element.

“Amino acid” is used herein to refer to either natural or synthetic amino acids, including glycine and D or L optical isomers, and amino acid analogs and peptidomimetics.

“Antibody” is used herein to refer to binding molecules including immunoglobulin molecules and immunologically active portions of immunoglobulin molecules, i.e., molecules that contain an antigen-binding site. Immunoglobulin molecules useful in the invention can be of any class (e.g., IgG, IgE, IgM, IgD, and IgA) or subclass. Native antibodies and immunoglobulins are usually heterotetrameric glycoproteins of about 150,000 daltons, composed of two identical light chains and two identical heavy chains. Each heavy chain has at one end a variable domain followed by a number of constant domains. Each light chain has a variable domain at one end and a constant domain at its other end. Antibodies include, but are not limited to, polyclonal, monoclonal, bispecific, chimeric, partially or fully humanized antibodies, fully human antibodies (i.e., generated in a transgenic mouse expressing human immunoglobulin genes), camel antibodies, and anti-idiotypic antibodies. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. The terms “antibody” and “immunoglobulin” are used interchangeably.

“Antibody fragment” or “antibody portion” are used herein to refer to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)₂, scFv, Fv, dsFv diabody, minibody, Fc fragments, and single chain antibodies. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

“Antigen-binding site” is used herein to refer to the variable domain of a heavy chain associated with the variable domain of a light chain.

“Bind” or “binding” are used herein to refer to detectable relationships or associations (e.g. biochemical interactions) between molecules.

“Cells” or “host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Comprise” and “comprising” are used in the inclusive, open sense, meaning that additional elements may be included.

“Interaction” refers to a physical association between two or more molecules, e.g., binding. The interaction may be direct or indirect.

“Inflammatory disease” is used herein to refer to a disease or disorder that is caused or contributed to by a complicated set of functional and cellular adjustments involving acute or chronic changes in microcirculation, movement of fluids, and influx and activation of inflammatory cells (e.g., leukocytes) and complement, and included autoimmune diseases. Examples of such diseases and conditions include, but are not limited to: reperfusion injury, ischemia injury, stroke, autoimmune hemolytic anemia, idiopathic thrombocytopenic purpura, rheumatoid arthritis, celiac disease, hyper-IgM immunodeficiency, arteriosclerosis, coronary artery disease, sepsis, myocarditis, encephalitis, transplant rejection, hepatitis, thyroiditis (e.g. Hashimoto's thyroiditis, Graves disease), osteoporosis, polymyositis, dermatomyositis, Type I diabetes, gout, dermatitis, alopecia areata, systemic lupus erythematosus, lichen sclerosis, ulcerative colitis, diabetic retinopathy, pelvic inflammatory disease, periodontal disease, arthritis, juvenile chronic arthritis (e.g. chronic iridocyclitis), psoriasis, osteoporosis, nephropathy in diabetes mellitus, asthma, pelvic inflammatory disease, chronic inflammatory liver disease, chronic inflammatory lung disease, lung fibrosis, liver fibrosis, rheumatoid arthritis, chronic inflammatory liver disease, chronic inflammatory lung disease, lung fibrosis, liver fibrosis, Crohn's disease, ulcerative colitis, burns, and other acute and chronic inflammatory diseases of the Central Nervous System (CNS; e.g. multiple sclerosis), gastrointestinal system, the skin and associated structures, the immune system, the hepato-biliary system, or any site in the body where pathology can occur with an inflammatory component.

An “isolated” molecule, e.g., an isolated IgM, refers to a condition of being separate or purified from other molecules present in the natural environment.

“Natural IgM” is used herein to refer to an IgM antibody that is naturally produced in a mammal (e.g., a human). They have a pentameric ring structure wherein the individual monomers resemble IgGs thereby having two light (κ or λ) chains and two heavy (μ) chains. Further, the heavy chains contain an additional C_{H}4 domain. The monomers form a pentamer by disulfide bonds between adjacent heavy chains. The pentameric ring is closed by the disulfide bonding between a J chain and two heavy chains. Because of its high number of antigen binding sites, a natural IgM antibody is an effective agglutinator of antigen. Production of natural IgM antibodies in a subject are important in the initial activation of B-cells, macrophages, and the complement system. IgM is the first immunoglobulin synthesized in an antibody response.

“Nucleic acid” is used herein to refer to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

“Operatively linked” is used herein to refer to a juxtaposition wherein the components so described are in a relationship permitting them to function in their intended manner. For example, a coding sequence is “operably linked” to another coding sequence when RNA polymerase will transcribe the two coding sequences into a single mRNA, which is then translated into a single polypeptide having amino acids derived from both coding sequences. The coding sequences need not be contiguous to one another so long as the expressed sequences ultimately process to produce the desired protein. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

“Peptide” is used herein to refer to a polymer of amino acids of relatively short length (e.g. less than 50 amino acids). The polymer may be linear or branched, it may comprise modified amino acids, and it may be interrupted by non-amino acids. The term also encompasses an amino acid polymer that has been modified; for example, disulfide bond formation, glycosylation, lipidation, acetylation, phosphorylation, or any other manipulation, such as conjugation with a labeling component.

“Promoter” is used herein to refer to a minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the of a polynucleotide sequence. Both constitutive and inducible promoters, are included in the invention (see e.g., Bitter et al., Methods in Enzymology 153:516-544, 1987). For example, when cloning in bacterial systems, inducible promoters such as pL of bacteriophage, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the nucleic acid sequences of the invention. Tissue-specific regulatory elements may be used. Including, for example, regulatory elements from genes or viruses that are differentially expressed in different tissues.

“Specifically binds” is used herein to refer to the interaction between two molecules to form a complex that is relatively stable under physiologic conditions. The term is used herein in reference to various molecules, including, for example, the interaction of an antibody and an antigen (e.g. a peptide). Specific binding can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater. Methods for determining whether two molecules specifically bind are well known and include, for example, equilibrium dialysis, surface plasmon resonance, and the like.

“Stringency hybridization” or “hybridizes under low stringency, medium stringency, high stringency, or very high stringency conditions” is used herein to describes conditions for hybridization and washing. Guidance for performing hybridization reactions can be found in Current Protocols in Molecular Biology, John Wiley & Sons, N.Y. (1989), 6.3.1-6:3.6, which is incorporated by reference. Aqueous and non-aqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified. Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences and the percent homology between two sequences is a function of the number of conserved positions shared by the sequences, taking into account the number of gaps, and the length of each gap, which need to be introduced for optimal alignment of the two sequences. The comparison of sequences and determination of percent identity and/or homology between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available on the world wide web with the extension gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available on the world wide web with the extension gcg.com), using a NWSgapdna CMP matrix and a gap weight of 40, 50, 60, 70; or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frame shift gap penalty of 5.

The percent identity and/or homology between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Treating” is used herein to refer to any treatment of, or prevention of, or inhibition of a disorder or disease in a subject and includes by way of example: (a) preventing the disease or disorder from occurring in a subject that may be predisposed to the disease or disorder, but has not yet been diagnosed as having it; (b) inhibiting the disease or disorder, i.e., arresting its progression; or (c) relieving or ameliorating the disease or disorder, i.e., causing regression. Thus, treating as used herein includes, for example, repair and regeneration of damaged or injured tissue or cells at the site of injury or prophylactic treatments to prevent damage, e.g., before surgery.

“Vector” as used herein refers to a nucleic acid molecule, which is capable of transporting another nucleic acid to which it has been operatively linked and can include a plasmid, cosmid, or viral vector. One type of preferred vector is an episome, i.e., a nucleic acid capable of extra-chromosomal replication. Preferred vectors are those capable of autonomous replication and/or expression of nucleic acids to which they are linked. Vectors may be capable of directing the expression of genes to which they are operatively linked. A vector may also be capable of integrating into the host DNA. In the present specification, “plasmid” and “vector” are used interchangeably as a plasmid (a circular arrangement of double stranded DNA) is the most commonly used form of a vector. However, the invention is intended to include such other forms of vectors which serve equivalent functions and which become known in the art subsequently hereto. Viral vectors include, e.g., replication defective retroviruses, adenoviruses and adeno-associated viruses.

### 6.2 Natural IgM Antibodies

The present invention is based, at least in part, on the identification of natural immunoglobulins (Ig), in particular natural IgMs, that bind to the N2 self peptide. Certain IgMs may be obtained from the hybridoma that has been deposited with the American Type Culture Collection and provided Accession Number PTA-3507.

The present invention provides an isolated antibody or fragment thereof that specifically binds to the N2 self-peptide of Mouse NMHC-IIB (592-603) (LMKNMDPLNDNV (N2; SEQ ID NO:17)), an ischemic antigen. The antibody is capable of activating complement, thereby inducing an immune response to the ischemic antigen. The antibody may thus be used as an agonist to mimic an immune response to ischemic antigen. Additionally, the isolated natural IgM antibody may be used to mimic inflammatory diseases or disorders such as ischemia-reperfusion injury when administered to animals, thereby creating animal models of such inflammatory diseases or disorders. The animal models may be used to test, screen, or identify treatments for such inflammatory diseases or disorders. In one aspect the animal model is an animal model of ischemia-reperfusion injury, which may be used for test, screen, or identify treatments for ischemia-reperfusion injury.

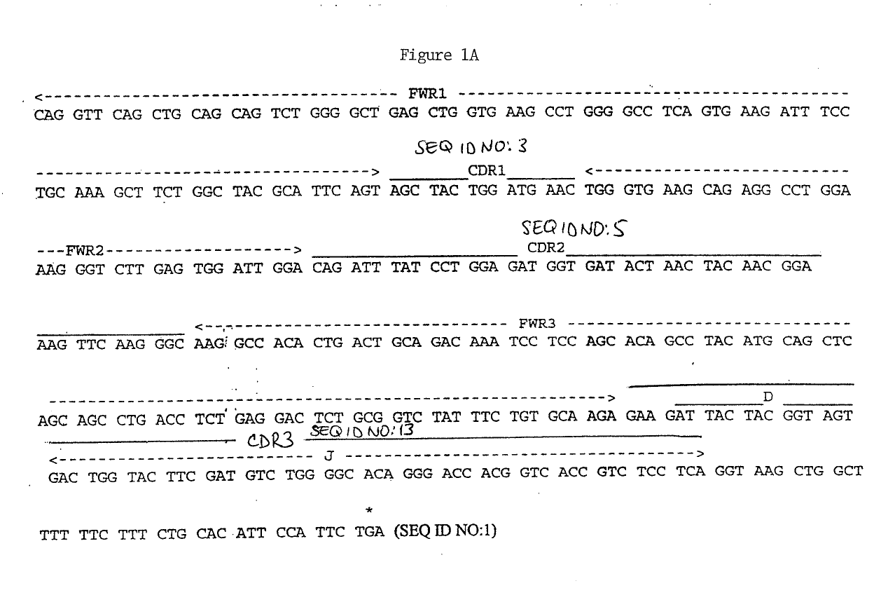

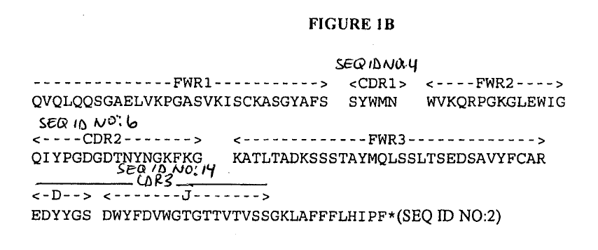

The present invention encompasses antibodies that immunospecifically bind to the N2 self-peptide having heavy chain variable region (“VH”) comprising one or more VH complementarity determining regions (“CDRs”) shown in FIGS. 1 and 2.

The nucleotide sequence of the heavy chain variable region of the IgM produced from hybridoma PTA-3507, IgM^{CM-22} (also referred to as 22A5 IgM) is shown in FIG. 1A (SEQ ID NO: 1), and the amino acid sequence is shown in FIG. 1B (SEQ ID NO: 2). The CDR1 domain of the heavy chain variable region corresponds to amino acids 31 to 35 of SEQ ID NO: 2 (SEQ ID NO: 4), which is encoded by nucleotides 91-105 of SEQ ID NO: 1 (SEQ ID NO: 3), and the CDR2 domain of the heavy chain variable region corresponds to amino acids 50 to 66 of SEQ ID NO: 2 (SEQ ID NO: 6), which is encoded by nucleotides 148-198 of SEQ ID NO: 1 (SEQ ID NO: 5). The CDR3 domain of the heavy chain variable region of SEQ ID NO: 2 is also shown in FIG. 1A as SEQ ID NO: 14, which is encoded by nucleotides of SEQ ID NO: 1 shown as SEQ ID NO: 13 in FIG. 1A.

The nucleotide sequence of the light chain variable region (“VL”) of IgM^{CM-22} is shown in FIG. 2 (SEQ ID NO: 7), and the amino acid sequence is shown in FIG. 2 (SEQ ID NO: 8). The CDR1 domain of the light chain variable region of SEQ ID NO: 8 is shown in FIG. 2 (SEQ ID NO: 10), which is encoded by nucleotides of SEQ ID NO: 7 as shown in FIG. 2 (SEQ ID NO: 9), and the CDR2 domain of the light chain variable region of SEQ ID NO: 8 is shown in FIG. 2 (SEQ ID NO: 12), which is encoded by nucleotides of SEQ ID NO: 7 as shown in FIG. 2 (SEQ ID NO: 11). The CDR3 domain of the light chain variable region of SEQ ID NO: 8 is also shown in FIG. 2 as SEQ ID NO: 16, which is encoded by nucleotides of SEQ ID NO: 7 shown as SEQ ID NO: 15 in FIG. 2.

The invention features polypeptides and fragments of the IgM^{CM-22} heavy chain variable regions and/or light chain variable regions. In exemplary embodiments, the isolated polypeptides comprise, for example, the amino acid sequences of SEQ ID NOs: 8, 10, 12, and/or 16, or fragments or combinations thereof; or SEQ ID NO: 2, 4, 6, and/or 14, or fragments or combinations thereof. The polypeptides of the present invention include polypeptides having at least, but not more than 20, 10, 5, 4, 3, 2, or 1 amino acid that differs from SEQ ID NOs: 8, 10, 12, 16, 2, 4, 6, or 14. Exemplary polypeptides are polypeptides that retain biological activity, e.g., the ability to bind the N2 self-peptide, and the ability to activate complement, thereby inducing an immune response to self antigen. In another embodiment, the polypeptides comprise polypeptides having at least 80%, 90%, 95%, 96%, 97%, 98%, and 99% sequence identity with a light chain variable region, or portion thereof, e.g. a light chain variable region polypeptide of SEQ ID NOs: 8, 10, 12, or 16. In another embodiment, the polypeptides comprise polypeptides having at least 80%, 90%, 95%, 96%, 97%, 98%, and 99% sequence identity with a heavy chain variable region, or portion thereof, e.g. a heavy chain variable region polypeptide of SEQ ID NOs: 2, 4, 6, or 14. In another embodiment, the invention features a polypeptide comprising the amino acid sequence of SEQ ID NO: 8 and SEQ ID NO: 2, further comprising an IRES sequence.

In one embodiment of the present invention, antibodies that immunospecifically bind to the N2 self-peptide comprise a VH CDR1 having the amino acid sequence of SEQ ID NO:4. In another embodiment, antibodies that immunospecifically bind to the N2 self-peptide comprise a VH CDR2 having the amino acid sequence of SEQ ID NO:6. In another embodiment, antibodies that immunospecifically bind to the N2 self-peptide comprise a VH CDR3 having the amino acid sequence of SEQ ID NO:14.

In another embodiment, antibodies that immunospecifically bind to the N2 self-peptide comprise a VH CDR1 having the amino acid sequence of SEQ ID NO:4 and a VH CDR2 having the amino acid sequence of SEQ ID NO:6. In another embodiment, antibodies that immunospecifically bind to the N2 self-peptide comprise a VH CDR1 having the amino acid sequence of SEQ ID NO:4 and a VH CDR3 having the amino acid of SEQ ID NO:14. In yet another embodiment, antibodies that immunospecifically bind to the N2 self-peptide comprise a VH CDR2 having the amino acid sequence of SEQ ID NO:6 and a VH CDR3 having the amino acid of SEQ ID NO:14. In another embodiment, antibodies that immunospecifically bind to the N2 self-peptide comprise a VH CDR1 having the amino acid sequence of SEQ ID NO:4, a VH CDR2 having the amino acid sequence of SEQ ID NO:6, and a VH CDR3 having the amino acid of SEQ ID NO:14.

The present invention encompasses antibodies that immunospecifically bind to the N2 self-peptide having a light chain variable region comprising one or more VL complementarity determining regions shown in FIGS. 1 and 2. In one embodiment of the present invention, antibodies that immunospecifically bind to an N2 self-peptide comprise a VL CDR1 having the amino acid sequence of SEQ ID NO:10. In another embodiment, antibodies that immunospecifically bind to the N2 self-peptide comprise a VL CDR2 having the amino acid sequence of SEQ ID NO:12. In another embodiment, antibodies that immunospecifically bind to the N2 self-peptide comprise a VL CDR3 having the amino acid sequence of SEQ ID NO:16.

In another embodiment, antibodies that immunospecifically bind to the N2 self-peptide comprise a VL CDR1 having the amino acid sequence of SEQ ID NO:10 and a VL CDR2 having the amino acid sequence of SEQ ID NO:12. In another embodiment, antibodies that immunospecifically bind to the N2 self-peptide comprise a VL CDR1 having the amino acid sequence of SEQ ID NO:10 and a VL CDR3 having the amino acid of SEQ ID NO:16. In another embodiment, antibodies that immunospecifically bind to the N2 self-peptide comprise a VL CDR2 having the amino acid sequence of SEQ ID NO:12 and a VL CDR3 having the amino acid of SEQ ID NO:16. In yet another embodiment, antibodies that immunospecifically bind to the N2 self-peptide comprise a VL CDR1 having the amino acid sequence of SEQ ID NO:10, a VL CDR2 having the amino acid sequence of SEQ ID NO:12, and a VL CDR3 having the amino acid of SEQ ID NO:16.

The present invention also provides antibodies comprising one or more VH CDRs and one or more VL CDRs as shown in FIGS. 1 and 2. In particular, the invention provides for an antibody comprising a VH CDR1 and a VL CDR1, a VH CDR1 and a VL CDR2, a VH CDR1 and a VL CDR3, a VH CDR2 and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof of the VH CDRs and VL CDRs shown in FIGS. 1 and 2.

In one embodiment, an antibody of the invention comprises a VH CDR1 having the amino acid sequence of SEQ ID NO:4 and a VL CDR1 having the amino acid sequence of SEQ ID NO:10. In another embodiment, an antibody of the present invention comprises a VH CDR1 having the amino acid sequence of SEQ ID NO:4 and a VL CDR2 having the amino acid sequence of SEQ ID NO:12. In another embodiment, an antibody of the present invention comprises a VH CDR1 having the amino acid sequence of SEQ ID NO:4 and a VL CDR3 having the amino acid sequence of SEQ ID NO:16.

In another embodiment, an antibody of the present invention comprises a VH CDR2 having the amino acid sequence of SEQ ID NO:6 and a VL CDR1 having the amino acid sequence of SEQ ID NO:10. In another embodiment, an antibody of the present invention comprises a VH CDR2 having the amino acid sequence of SEQ ID NO:6 and a VL CDR2 having the amino acid sequence of SEQ ID NO:12. In another embodiment, an antibody of the present invention comprises a VH CDR2 having the amino acid sequence of SEQ ID NO:6 and a VL CDR3 having the amino acid sequence of SEQ ID NO:16.

In another embodiment, an antibody of the present invention comprises a VH CDR3 having the amino acid sequence of SEQ ID NO:14, and a VL CDR1 having the amino acid sequence of SEQ ID NO:10. In another embodiment, an antibody of the present invention comprises a VH CDR3 having the amino acid sequence of SEQ ID NO:14 and a VL CDR2 having the amino acid sequence of SEQ ID NO:12. In a preferred embodiment, an antibody of the present invention comprises a VH CDR3 having the amino acid sequence of SEQ ID NO:14 and a VL CDR3 having the amino acid sequence of SEQ ID NO:16.

In certain embodiments, the V region domains of heavy and light chains can be expressed on the same polypeptide, joined by a flexible linker to form a single-chain Fv fragment, and the scFV gene subsequently cloned into the desired expression vector or phage genome. As generally described in McCafferty et al., Nature (1990) 348:552-554, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with the antigen can subsequently be formulated into a pharmaceutical preparation for use in the subject method.

An antibody of the present invention can be one in which the variable region, or a portion thereof, e.g., the complementarity determining regions (CDR or CDRs), are generated in a non-human organism, e.g., a rat or mouse. Chimeric, CDR-grafted, and humanized antibodies are within the invention. Any modification is within the scope of the invention so long as the antibody has at least one antigen binding portion.

Chimeric antibodies (e.g. mouse-human monoclonal antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding another Fc constant region is substituted. (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl. Cancer Inst. 80:1553-1559).

A chimeric antibody can be further made by replacing sequences of the Fv variable region which are not directly involved in antigen binding with equivalent sequences from another Fv variable region.

CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare antibodies of the present invention (UK Patent Application GB 2188638A, filed on Mar. 26, 1987; Winter U.S. Pat. No. 5,225,539), the contents of which is expressly incorporated by reference.

A CDR-grafted antibody will have at least one or two but generally all recipient CDRs (of heavy and/or light immunoglobulin chains) replaced with a donor CDR. Preferably, the donor will be a rodent antibody, e.g., a rat or mouse antibody, and the recipient will be a different framework region or a consensus framework region. Typically, the immunoglobulin providing the CDRs is called the “donor” and the immunoglobulin providing the framework is called the “acceptor.” In one embodiment, the donor immunoglobulin is a non-human (e.g., rodent). The acceptor framework can be a naturally-occurring (e.g., a human) framework or a consensus framework, or a sequence about 85% or higher, preferably 90%, 95%, 99% or higher identical thereto.

All of the CDRs of a particular antibody may be replaced with a portion of another CDR or only some of the CDRs may be replaced with other CDRs.

Also within the scope of the invention are chimeric antibodies in which specific amino acids have been substituted, deleted or added. In particular, antibodies may have amino acid substitutions in the framework region, such as to improve binding to the antigen. For example, an antibody will have framework residues identical to the donor framework residue or to another amino acid other than the recipient framework residue. As another example, in an antibody having mouse CDRs, amino acids located in the human framework region can be replaced with the amino acids located at the corresponding positions in another antibody. Such substitutions are known to improve binding of antibodies to the antigen in some instances.

Antibody fragments of the invention are obtained using conventional procedures known to those with skill in the art. For example, digestion of an antibody with pepsin yields F(ab′)2 fragments and multiple small fragments. Mercaptoethanol reduction of an antibody yields individual heavy and light chains. Digestion of an antibody with papain yields individual Fab fragments and the Fc fragment.

In another aspect, the invention also features a modified natural immunoglobulin, e.g., which functions as an agonist (mimetic). Preferably the modified natural immunoglobulin, e.g., modified pathogenic immunoglobulin, functions as an agonist of complement activation. Variants of the pathogenic immunoglobulin can be generated by mutagenesis, e.g., discrete point mutation, the insertion or deletion of sequences or the truncation of a pathogenic immunoglobulin. An agonist of the natural immunoglobulin can retain substantially the same, or a subset, of the biological activities of the naturally occurring form of the protein. An agonist of a natural immunoglobulin can mimic one or more of the activities of the naturally occurring form of the pathogenic immunoglobulin by, for example, being capable of binding to an ischemic specific antigen, and capable of activating a complement pathway.

Variants of a natural immunoglobulin can be identified by screening combinatorial libraries of mutants, e.g., truncation mutants, of a natural immunoglobulin for agonist or antagonist activity.

Libraries of fragments e.g., N terminal, C terminal, or internal fragments, of a natural immunoglobulin coding sequence can be used to generate a variegated population of fragments for screening and subsequent selection of variants of this protein. Variants in which a cysteine residue is added or deleted or in which a residue that is glycosylated is added or deleted are particularly preferred.

### 6.3 Isolated Nucleic Acids Encoding IgM Antibodies

Isolated nucleic acids encoding each antibody described above is provided. The nucleic acid compositions of the present invention, while often in a native sequence (except for modified restriction sites and the like), from either cDNA, genomic or mixtures may be mutated, in accordance with standard techniques. For coding sequences, these mutations, may affect the amino acid sequence as desired. In particular, nucleotide sequences substantially identical to or derived from native V, D, J, constant, switches and other such sequences described herein are contemplated. Due to the degeneracy of the genetic code, other nucleotide sequences can encode the amino acid sequences listed herein.

For example, an isolated nucleic acid can comprise an IgM^{CM-22} (or 22A5 IgM) heavy chain variable region nucleotide sequence having a nucleotide sequence as shown in FIG. 1A (SEQ ID NO: 1), or a sequence, which is at least 80%, 90%, 95%, 96%, 97%, 98%, or 99% identical to SEQ ID NO: 1. A nucleic acid molecule may comprise the heavy chain CDR1 nucleotide sequence of SEQ ID NO: 3, or a portion thereof, or a sequence, which is at least 80%, 90%, 95%, 96%, 97%, 98%, or 99% identical to SEQ ID NO: 3. Further, the nucleic acid molecule may comprise the heavy chain CDR2 nucleotide sequence of SEQ ID NO: 5, or a portion thereof, or a sequence, which is at least 80%, 90%, 95%, 96%, 97%, 98%, or 99% identical to SEQ ID NO: 5. In an exemplary embodiment, the nucleic acid molecule comprises a heavy chain CDR1 nucleotide sequence of SEQ ID NO: 3, or portion thereof, and a heavy chain CDR2 nucleotide sequence of SEQ ID NO: 5, or portion thereof. The nucleic acid molecules of the present invention may comprise heavy chain sequences, e.g. SEQ ID NO: 1, SEQ ID NO: 3, SEQ ID NO: 5, or combinations thereof, or encompass nucleotides having at least 80%, 90%, 95%, 96%, 97%, 98%, and 99% sequence identity to SEQ ID NOs: 1, 3 or 5. Further, the nucleic acid molecules of the present invention may comprise heavy chain sequences, which hybridize under stringent conditions, e.g. low, medium, high or very high stringency conditions, to SEQ ID NOs: 1, 3 or 5.

In another embodiment, the invention features nucleic acid molecules having at least 80%, 90%, 95%, 96%, 97%, 98%, and 99% sequence identity with a nucleic acid molecule encoding a heavy chain polypeptide, e.g., a heavy chain polypeptide of SEQ ID NOs: 2, 4 or 6. The invention also features nucleic acid molecules which hybridize to nucleic acid sequences encoding a heavy chain variable region of a natural antibody or portion thereof, e.g., a heavy chain variable region of SEQ ID NO: 2, 4 or 6.

In another embodiment, the isolated nucleic acid encodes a IgM^{CM-22} (22A5 IgM) light chain variable region nucleotide sequence having a nucleotide sequence as shown in FIG. 2 (SEQ ID NO: 7), or a sequence at least 80%, 90%, 95%, 96%, 97%, 98%, 99% identical to SEQ ID NO: 7. The nucleic acid molecule may comprise the light chain CDR1 nucleotide sequence of SEQ ID NO: 9, or a portion thereof, or a sequence, which is at least 80%, 90%, 95%, 96%, 97%, 98%, or 99% identical to SEQ ID NO: 9. In another preferred embodiment, the nucleic acid molecule may comprise the light chain CDR2 nucleotide sequence of SEQ ID NO: 11, or a portion thereof, or a sequence, which is at least 80%, 90%, 95%, 96%, 97%, 98%, or 99% identical to SEQ ID NO: 11. In another preferred embodiment, the nucleic acid molecule may comprise the light chain CDR3 nucleotide sequence of SEQ ID NO: 7 (SEQ ID NO: 15) as shown in FIG. 2, or a portion thereof, or a sequence, which is at least 80%, 90%, 95%, 96%, 97%, 98%, or 99% identical to SEQ ID NO: 15. In an exemplary embodiment, the nucleic acid molecule comprises a light chain CDR1 nucleotide sequence of SEQ ID NO: 9, or portion thereof, and a light chain CDR2 nucleotide sequence of SEQ ID NO: 11, or portion thereof. The nucleic acid molecules of the present invention may comprise light chain sequences, e.g. SEQ ID NOs: 7, 9 or 11, or combinations thereof, or encompass nucleotides having at least 80%, 90%, 95%, 96%, 97%, 98%, and 99% sequence identity to SEQ ID NOs: 7, 9 or 11. Further nucleic acid molecules may comprise light chain sequences, which hybridize under stringent conditions, e.g. low, medium, high or very high stringency conditions, to SEQ ID NOs: 7, 9 or 11.

Nucleic acid molecules can have at least 80%, 90%, 95%, 96%, 97%, 98% or 99% sequence identity with a nucleic acid molecule encoding a light chain polypeptide, e.g., a light chain polypeptide of SEQ ID NOs: 8, 10, or 12. The invention also features nucleic acid molecules which hybridize to a nucleic acid sequence encoding a light chain variable region of a natural antibody or portion thereof, e.g., a light chain variable region of SEQ ID NOs: 8, 10 or 12.

In another embodiment, the invention provides an isolated nucleic acid encoding a heavy chain CDR1 domain comprising the amino acid sequence of SEQ ID NO: 4, or a fragment or modified form thereof. This nucleic acid can encode only the CDR1 region or can encode an entire antibody heavy chain variable region or a fragment thereof. For example, the nucleic acid can encode a heavy chain variable region having a CDR2 domain comprising the amino acid sequence of SEQ ID NO: 6. In yet another embodiment, the invention provides an isolated nucleic acid encoding a heavy chain CDR2 domain comprising the amino acid sequence of SEQ ID NO: 6, or a fragment or modified form thereof. This nucleic acid can encode only the CDR2 region or can encode an entire antibody heavy chain variable region or a fragment thereof. For example, the nucleic acid can encode a light chain variable region having a CDR1 domain comprising the amino acid sequence of SEQ ID NO: 4.

In still another embodiment, the invention provides an isolated nucleic acid encoding a light chain CDR1 domain comprising the amino acid sequence of SEQ ID NO: 10, or a fragment or modified form thereof. This nucleic acid can encode only the CDR1 region or can encode an entire antibody light chain variable region. For example, the nucleic acid can encode a light chain variable region having a CDR2 domain comprising the amino acid sequence of SEQ ID NO: 12. The isolated nucleic acid can also encode a light chain CDR2 domain comprising the amino acid sequence of SEQ ID NO: 12, or a fragment or modified form thereof. This nucleic acid can encode only the CDR2 region or can encode an entire antibody light chain variable region. For example, the nucleic acid can encode a light chain variable region having a CDR1 domain comprising the amino acid sequence of SEQ ID NO: 10.

The nucleic acid encoding the heavy or light chain variable region can be, for example, of murine or human origin, or can comprise a combination of murine and human amino acid sequences. For example, the nucleic acid can encode a heavy chain variable region comprising the CDR1 of SEQ ID NO: 2 (SEQ ID NO: 4) and/or the CDR2 of SEQ ID NO: 2 (SEQ ID NO: 6), and another (e.g. human) framework sequence. In addition, the nucleic acid can encode a light chain variable region comprising the CDR1 of SEQ ID NO: 8 (SEQ ID NO: 10) and/or the CDR2 of SEQ ID NO: 8 (SEQ ID NO: 12), and another (e.g. human) framework sequence. The invention further encompasses vectors containing the above-described nucleic acids and host cells containing the expression vectors.

Methods for screening gene products of combinatorial libraries made by point mutations or truncation, and for screening cDNA libraries for gene products having a selected property. Recursive ensemble mutagenesis (REM), a technique which enhances the frequency of functional mutants in the libraries, can be used in combination with the screening assays to identify variants (Arkin and Yourvan (1992) Proc. Natl. Acad. Sci. USA 89:7811-7815; Delgrave et al. (1993) Protein Engineering 6(3):327-331).

Cell based assays can be exploited to analyze a variegated library. For example, a library of expression vectors can be transfected into a cell line, e.g., a cell line, which ordinarily responds to the protein in a substrate-dependent manner. Plasmid DNA can then be recovered from the cells which score for inhibition, or alternatively, potentiation of signaling by the pathogenic immunoglobulin-substrate, and the individual clones further characterized.

### 6.4 Methods

In a further aspect, the invention features methods of inducing an immune response to an N2 self-peptide in a non-human animal by administering to the animal a natural antibody described herein. Administration of the antibody to the non-human animal mimics inflammatory diseases or disorders in the animal such as ischemia-reperfusion injury, thereby creating animal models of such inflammatory diseases or disorders.

The invention also features a method of making a natural immunoglobulin, e.g., a pathogenic immunoglobulin having a non-wild type activity, e.g., an antagonist, agonist, or super agonist of a naturally occurring pathogenic immunoglobulin. The method includes: altering the sequence of a natural immunoglobulin disclosed herein, e.g., by substitution or deletion of one or more residues of a non-conserved region, a domain or residue disclosed herein, and testing the altered polypeptide for the the ability to specifically bind the N2 self-peptide and activate complement, thereby, thereby inducing an immune response to self antigen.

Further, the invention features a method of making a fragment or analog of a natural immunoglobulin, e.g., a pathogenic immunoglobulin having an altered biological activity of a naturally occurring pathogenic immunoglobulin. The method includes: altering the sequence, e.g., by substitution or deletion of one or more residues, of a pathogenic immunoglobulin, e.g., altering the sequence of a non-conserved region, or a domain or residue described herein, and testing the altered polypeptide for the desired activity. In an exemplary embodiment, the modified natural immunoglobulin may have a reduced ability to activate complement. For example, one or more of the amino acid residues involved in complement binding and/or activation are mutated.

In certain embodiment, the modified natural antibody may comprise at least the CDR1 region of SEQ ID NO: 8 (SEQ ID NO: 10), or antigen binding portions thereof, and/or at least the CDR2 region of SEQ ID NO: 8 (SEQ ID NO: 12), or antigen binding portions thereof, and/or at least the CDR3 region of SEQ ID NO:8, or antigen binding portions thereof. In another embodiment, the modified antibody may comprise at least the CDR1 region of SEQ ID NO: 2 (SEQ ID NO: 4), or antigen binding portions thereof, and/or at least the CDR2 region of SEQ ID NO: 2 (SEQ ID NO: 6), or antigen binding portions thereof, and/or at least the CDR3 region of SEQ ID NO:2, or antigen binding portions thereof. In an exemplary embodiment, the modified antibody comprises the CDR1 region of SEQ ID NO: 8 (SEQ ID NO: 10), the CDR2 region of SEQ ID NO: 8 (SEQ ID NO: 12), and the CDR3 region of SEQ ID NO: 8 or antigen binding portions thereof. In another exemplary embodiment, the modified antibody comprises the CDR1 region of SEQ ID NO: 2 (SEQ ID NO: 4), the CDR2 region of SEQ ID NO: 2 (SEQ ID NO: 6), and the CDR3 region of SEQ ID NO: 2 or antigen binding portions thereof. The modified antibody may also comprise the CDR1 region of SEQ ID NO: 8 (SEQ ID NO: 10) and the CDR2 region of SEQ ID NO: 8 (SEQ ID NO: 12) and the modified antibody comprises the CDR1 region of SEQ ID NO: 2 (SEQ ID NO: 4) and the CDR2 region of SEQ ID NO: 2 (SEQ ID NO: 6) or antigen binding portions thereof.

The modified natural antibody is an antibody having a binding affinity to the ischemic-specific antigen, similar, e.g., greater than, less than, or equal to, the binding affinity of the antibody produced by the hybridoma deposited with the ATCC, having the accession number PTA-3507. In another embodiment, the natural antibody can be a non-human antibody, e.g., a cow, goat, mouse, rat, sheep, pig, or rabbit. In an exemplary embodiment, the non-human antibody is a murine antibody. The natural antibody may also be a recombinant antibody. The modified natural antibody may be an IgG or IgM antibody. In another embodiment, the isolated natural immunoglobulin possess the same antigenic specificity as the immunoglobulin produced by the hybridoma deposited with the ATCC, having accession number PTA-3507.

### Exemplification

The invention, having been generally described, may be more readily understood by reference to the following examples, which are included merely for purposes of illustration of certain aspects and embodiments of the present invention, and are not intended to limit the invention in any way.

### EXAMPLE 1

### Mechanism of Ischemia-Reperfusion Injury

This Example shows that mice deficient in the complement system were resistant to ischemia-reperfusion injury.

To examine the mechanism of ischemia-reperfusion injury, mice deficient in complement C3 were treated in the hindlimb model. The C3−/− mice were partially protected from injury based on an approximate 50% reduction in permeability index (see Weiser et al. (1996) J. Exp. Med. 1857-1864). Thus, complement C3 is essential for induction of full injury in this murine model.

The experiments in Weiser et al. did not identify how complement was activated. The serum complement system can be activated by at least three distinct pathways, classical, lectin or alternative. Knowing which pathway is involved, is important as it suggests a mechanism for injury. For example, the classical pathways is activated very efficiently by IgM and IgG isotypes of immunoglobulin or by the serum recognition protein C-reactive protein. Whereas, the lectin pathway is activated following recognition of specific carbohydrates such as mannan by mannan binding lectin (MBL) (Epstein et al., (1996) Immunol 8, 29-35). In both pathways, complement C4 is required in forming an enzyme complex with C2 that catalyzes cleavage of the central component C3. By contrast, the alternative pathway activates spontaneously leading to conversion of C3 to its active form (C3b) and attachment to foreign-or self-tissues. The pathway is tightly regulated as all host cells express inhibitors of amplification of the complement pathway by inactivating, or displacing the C3 convertase (Muller-Eberhard, H. J., (1988) Ann. Rev. Biochem. 57, 321-347). One approach for determining the pathway involved is use of mice deficient in C4, i.e., cannot form C3 convertase via classical or lectin pathways. Comparison of mice deficient in either C3 or C4 with wild type (WT) controls in the hindlimb model, revealed that C4 was also required for induction of full injury (Weiser et al. supra). This finding was important as it suggested that antibody or MBL might be involved.

### EXAMPLE 2

### Natural IgM Mediates Ischemia Reperfusion (I/R) Injury

This Example shows that mice deficient in immunoglobulin were resistant to ischemia-reperfusion injury.

To determine if antibody was involved in mediating I/R injury, mice totally deficient in immunoglobulin, RAG2−/− (recombinase activating gene-2 deficient) were characterized along with the complement deficient animals in the intestinal model. Significantly, the RAG-2−/− mice were protected to a similar level as observed in the complement deficient animals (Weiser et al. supra). Since the RAG2−/− animals are also missing mature lymphocytes, it was important to determine that the pathogenic effect was antibody dependent (Shinkai et al. (1992) Cell 68, 855-867). To confirm that injury was mediated by serum antibody, the deficient animals were reconstituted with either normal mouse sera (Weiser et al. supra) or purified IgM (Williams et al. (1999) J. Appl. Physiol 86; 938-42). In both cases, the reconstituted RAG-2−/− mice were no longer protected and injury was restored. In the latter experiments, a model of intestinal injury was used as in this model, injury is thought to be mediated primarily by complement.

The interpretation of these results is that during the period of ischemia, neoantigens are either expressed or exposed on the endothelial cell surface. Circulating IgMs appear to recognize the new determinant, bind and activate classical pathway of complement. While the nature of the antigen is not known, IgM rather than IgG seems to be primarily responsible for activation of complement as reconstitution of deficient mice with pooled IgG did not significantly restore injury in the mice. An alternative hypothesis is that there is another initial event such as the MBL pathway that recognizes the altered endothelial surface, induces low level complement activation which in turn exposes new antigenic sites and the pathway is amplified by binding of IgM.

### EXAMPLE 3

### Pathogenic IgM is a Product of B-1 Cells

Since a major fraction of circulating IgM is thought to represent natural antibody, i.e. product of rearranged germline genes, it is possible that mice bearing deficiencies in the B-1 fraction of lymphocytes might also be protected. B-1 cells have a distinct phenotype from more conventional B-2 cells in that they express low levels of IgD and CD23 and a major fraction express the cell surface protein CD5 (Hardy et al., (1994) Immunol. Rev.: 137, 91; Kantor et al. (1993) Annu. Rev. Immunol. 11, 501-538, 1993. B-1 cells are also distinguished by reduced circulation in mice, limited frequency in the peripheral lymph nodes and spleen and are primarily localized within the peritoneal cavity. To examine a role for B-1 cells as a source of pathogenic IgM, antibody-deficient mice (RAG-2−/−) were reconstituted with 5×10⁵ peritoneal B-1 cells and rested approximately 30 days before treatment. Circulating IgM levels reach a near normal range within a month following adoptive transfer. Characterization of the B-1 cell reconstituted mice in the intestinal ischemia model confirmed that B-1 cells were a major source of pathogenic IgM (see Williams et al. (1999) supra). This was an important observation because the repertoire of B-1 cell natural antibody is considerably more limited than would be expected for conventional B-2 cells. Therefore, it is possible that the pathogenic antibody represents a product of the germline.

### EXAMPLE 4

### Cr2−/− Mice are Protected from Ischemia Reperfusion Injury

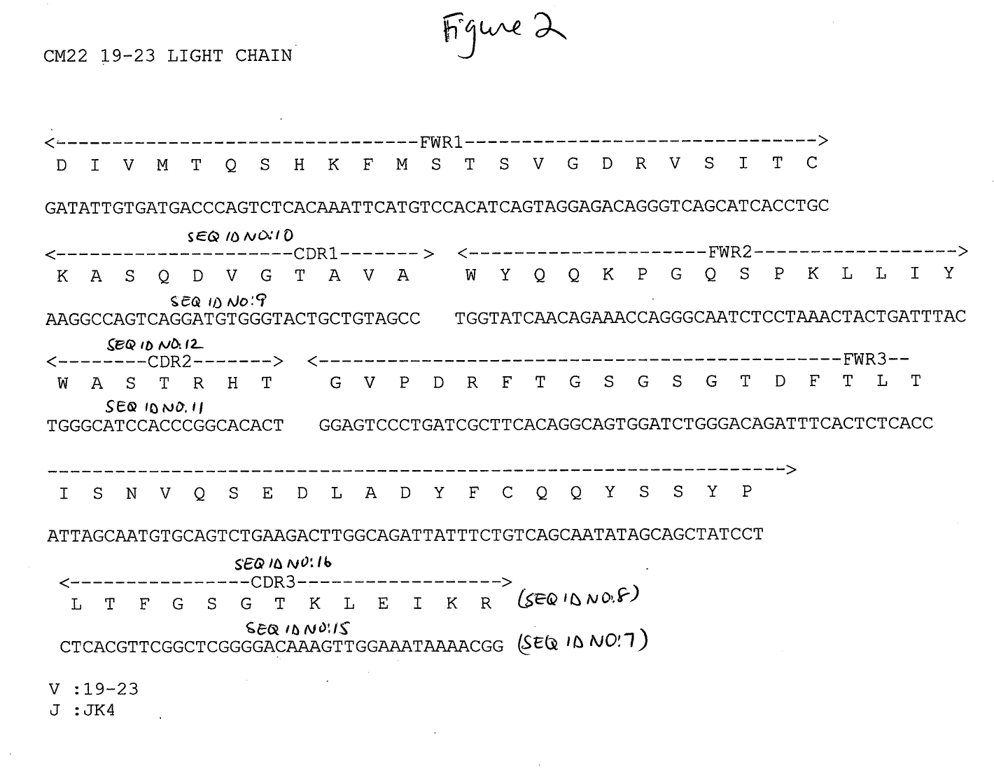

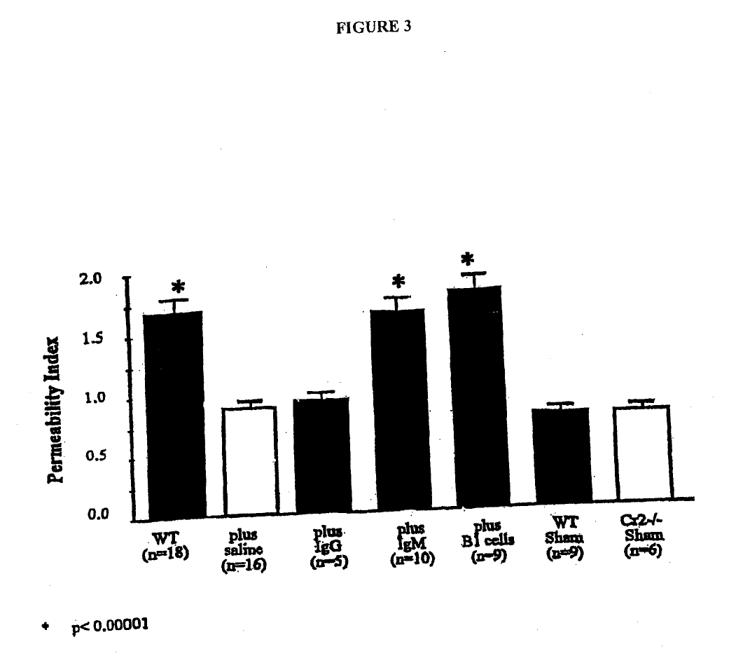

The initial characterization of Cr2−/− knockout mice revealed an approximate 50% reduction in the frequency of B-1a or CD5+B-1 cells (Ahearn et al. (1996) Immunity 4: 251-262). Although characterization of another strain of Cr2-deficient mice did not identify a similar reduction (Molina et al. (1996) Proc. Natl. Acad. Sci. USA 93, 3357-3361). Whether the difference in frequency of CD5+cells was due to variation in strain background or environmental differences is not known. Despite the reduced frequency of B-1 a cells in the Cr2−/− mice, circulating levels of IgM were within the normal range. These findings suggested that the repertoire of IgM might be different in the Cr2-deficient animals. To test this hypothesis, mice in the intestinal I/R model were characterized. Surprisingly, the Cr2−/− mice were equally protected as the complete-antibody deficient mice (FIG. 3). Comparison of survival over a five-day period following treatment in the intestinal model demonstrated a significant increase in mortality of the WT compared to Cr2-deficient animals. Consistent with an increased mortality, a dramatic reduction in injury was observed in tissue sections harvested from treated WT or Cr2−/− deficient mice.

Extensive injury to the mucosal layer of the intestine was observed in WT mice or Cr2−/− mice reconstituted with pooled IgM or B-1 cells. By contrast, tissue sections isolated from treated Cr2−/− mice were similar to that of sham controls. Thus, despite normal circulating levels of IgM, the Cr2-deficient mice were protected from injury. These results not only confirm the importance of B-1 cells as a source of pathogenic antibody but suggest that the complement system is somehow involved in formation or maintenance of the repertoire of natural antibody. For example, complement may be involved in positive selection of B-1 cells.

### EXAMPLE 5

### Identification of Pathogenic IgMs

This Example describes the generation of a specific hybridoma clone from normal B-1 cells and the identification of one clone that produces a pathogenic IgM. The pathogenic IgM was shown to restore injury in vivo to antibody deficient mice.

Studies in mice bearing a deficiency in complement receptors CD21/CD35, revealed that the mice were missing the pathogenic antibody. This finding was unexpected because they have a normal level of IgM in their blood. These findings led to the hypothesis that a special population of B cells termed B-1 cells are responsible for secreting the pathogenic IgM. For example, engraftment of the receptor deficient mice (Cr2−/−) with B-1 cells from normal mice restored injury, confirming the importance of B-I cells. To identify the specific antibody or antibodies responsible for injury, a panel of hybridoma clones were constructed from an enriched pool of peritoneal B-1 cells harvested from normal mice. The general approach for preparing hybridomas from enriched fraction of peritoneal cells includes harvesting peritoneal cells from mice treated 7 days earlier with IL-10 and subsequently enriched for CD23 negative B cells by negative selection with magnetic beads. Enriched B cells are analyzed by FACS following staining with IgM, Mac-1 and CD23 specific Mab. The enriched population is further activated by culturing with LPS for 24 hours. Activated cells are hybridized with fusion partner myeloma cells in the presence of PEG and grown in HAT-selective medium. Hybridomas are screened for IgM secreting clones by ELISA, and positive wells are expanded for purification of IgM.

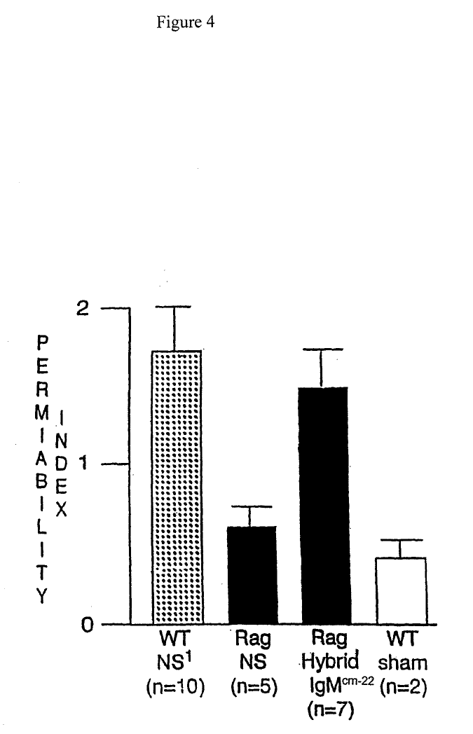

Twenty-two IgM-secreting hybridoma clones were analyzed by pooling an equal amount of IgM product from each of the clones. Treatment of antibody-deficient mice with the pooled IgM restored injury similar to that seen with pooled IgM from serum. This finding confirmed that the pathogenic IgM was among the twenty-two hybridomas produced. By dividing the pools into two fractions, i.e., 1-11 and 12-22, and treatment mice with the two fractions, the pathogenic antibody was found to fractionate with the pool that included clone # 22. Finally, mice were reconstituted with either clone 17 or 22. Clone 22 restored injury whereas the other clones did not (see FIG. 4).

### EXAMPLE 6

### Complement Involvement in B-1 Cell Selection

Two different models have been proposed to explain the development of B-1 cells. The lineage hypothesis proposes that B-1 cells develop in early fetal life as a distinct population (Kantor et al. (1993) supra). Alternatively, B-1 cells develop from the same progenitors as conventional B cells but depending on their environment, i.e., encounter with antigen, they develop into B-1 or retain the B-2 cell phenotype (Wortis, H. H. (1992) Int. Rev. Immunol. 8, 235; Clarke, J. (1998) Exp. Med. 187, 1325-1334). Irrespective of their origin, it is known that B-1 cells are not replenished from adult bone marrow at the same frequency as B-2 cells and that their phenotype is more similar to that of early fetal liver B cells or neonatal bone marrow (BM) cells. Consistent with an early origin, their repertoire tends to be biased towards expression of more proximal V_{H} genes and N-nucleotide addition is limited (Gu et al. (1990) EMBO J 9, 2133; Feeney, J. (1990) Exp. Med. 172, 1377). It seems reasonable that given the reduced replenishment by adult BM stem cells, B-1 cells are self-renewed and that antigen stimulation might be important in their renewal, expansion or even initial selection (Hayakawa et al., (1986) Eur. J. Immunol. 16, 1313). Indeed inherent to the conventional model, B-1 cells must be antigen selected.

Evidence in support of a B-cell receptor (BCR) signaling requirement for positive selection of B-1 cells comes from mice bearing mutations that alter BCR signaling. For example, impairment of BCR signaling through CD 19, vav, or Btk dramatically affects development of B-1 cells. By contrast, loss of negative selection such as in CD22- or SHIP-1 deficient mice can lead to an increase in B-1 cell frequency (O'Keefe et al. (1996) Science 274, 798-801; Shultz et al. (1993) Cell 73, 1445). Recent, elegant studies with mice bearing two distinct Ig transgenes, V_{H}12 (B-1 cell phenotype) or V_{H}B1-8 (B-2 cell phenotype) support the view that B-1 cells are positively selected by self-antigens. For example, B cells expressing V_{H}12 either alone or together with B1-8 developed a B-1 cell phenotype. Whereas, few if any B cells were identified that expressed the B1-8 transgene only. Thus, these results suggested that encounter of transgenic B cells with self-PtC resulted in expansion of those expressing V_{H} 12. Selection of B-1 cells was recently reported by Hardy et al. (1994) Immunol. Rev. 137, 91). In their model, B cells expressing an immunoglobulin transgene specific for Thy 1.1 were selected and expanded in mice expressing the cognate antigen. By contrast, transgene+B-1 cells were not found in mice that expressed the alternative allotype Thy 1.2.

Where does complement fit into B-1 cell development? The overall reduction in B-1a cell frequency and the more specific loss of B-1 cells expressing IgM involved in I/R injury suggests a role for CD21/CD35 in either positive selection or maintenance of B-1a cells. One possible role for complement is that it enhances BCR signaling on encounter with cognate antigen. Biochemical studies and analysis of CD21/CD35 deficient mice demonstrate the importance of co-receptor signaling in activation and survival of conventional B cells (Carroll, M. C., (1998) Ann. Rev. Immunol. 16, 545-568; Fearon et al. (1995) Annu. Rev. Immunol. 13, 127-149). It is very likely that B-1 cells likewise utilize co-receptor signaling to enhance the BCR signal. For example, bacteria express typical B-1 cell antigens such as phosphoryl choline and it is not unreasonable that coating of bacteria with complement ligand C3d would enhance crosslinking of the co-receptor with the BCR and enhance overall signaling. Thus, antigens expressed at lower concentrations might require complement enhancement in order for the cognate B-cell to recognize it and expand or be positively selected. Another role for complement receptors is in localizing antigen on follicular dendritic cells (FDC) within the lymphoid compartment. However, since the major population of B-1 cells occupy the peritoneal tissues it is not clear if they would encounter FDC within lymphoid structures. The actual site or sites in which B-1 cells undergo positive selection are not known. It is possible that they must encounter cognate antigen in early fetal development or in neonatal BM. If this is the case, it might be expected that complement receptors on stromal cells within these compartments bind antigen for presentation to B cells. It is possible that complement receptors could participate in both stages of development. First, they might enhance antigens signaling in initial positive selection. Secondly, as selected B-1 cells are replenished at peripheral sites, complement receptors might again be involved in enhancement of BCR signaling.

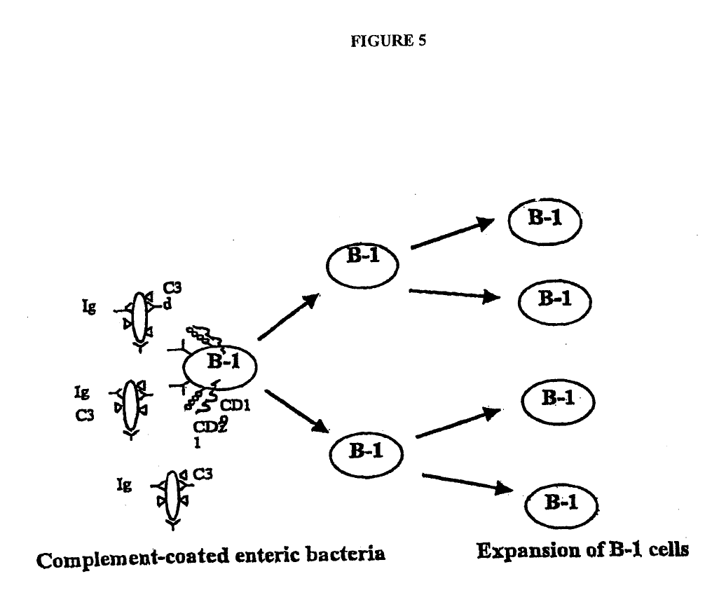

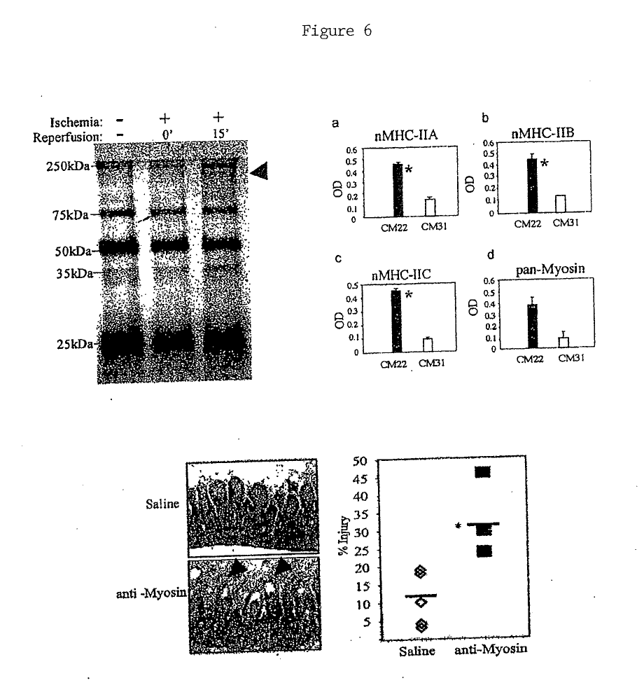

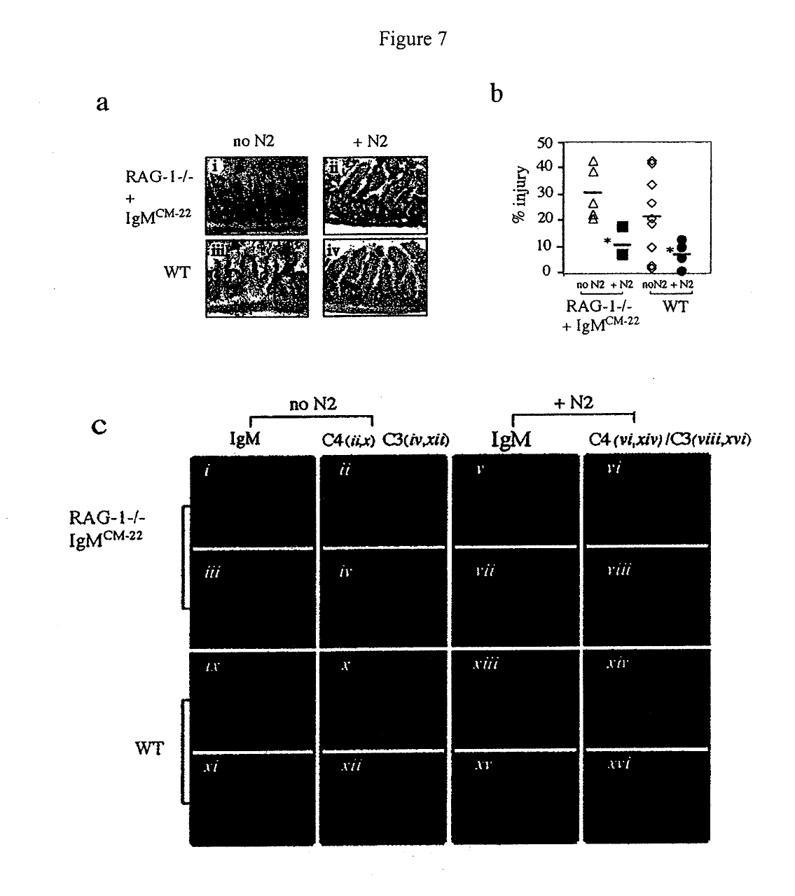

FIG. 5 is a schematic diagram of the proposed role for complement and complement receptors in positive selection of peritoneal B-1 lymphocytes. The interaction of complement-ligand coated antigens (self- and non-self) results in co-ligation of the CD21/CD19 co-receptor and BCR on the cell surface leading to enhanced signaling and positive selection.

### Incorporation by Reference

All publications, patents, and patent applications mentioned herein are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequence which reference an accession number correlating to an entry in a public database, such as those maintained by The Institute for Genomic Research (TIGR) on the world wide web with the extension tigr.org and or the National Center for Biotechnology Information (NCBI) on the world wide web with the extension ncbi.nlm.nih.gov.

### Equivalents

Those skilled in the art will recognize, or be able to ascertain using no more than routine experimentation, many equivalents to the specific embodiments of the invention described herein. Such equivalents are intended to be encompassed by the following claims.

1. An isolated antibody or antigen binding fragment thereof which specifically binds to an ischemic antigen, wherein the antibody or antigen binding fragment thereof comprises:
a) an amino acid sequence comprising the variable heavy (VH) complementary determining region (CDR) 1 of SEQ ID NO:2;
b) an amino acid sequence comprising the VH CDR2 of SEQ ID NO:2;
c) an amino acid sequence comprising the VH CDR3 of SEQ ID NO:2;
d) an amino acid sequence comprising the variable light (VL) CDR1 of SEQ ID NO:8;
e) an amino acid sequence comprising the VL CDR2 of SEQ ID NO:8; and
f) an amino acid sequence comprising the VL CDR3 of SEQ ID NO:8.

2. The isolated antibody or antigen binding fragment thereof of claim 1, wherein the antigen binding fragment is a Fab fragment.

3. The isolated antibody or antigen binding fragment thereof of claim 1, wherein the antigen binding fragment is a F(ab′)₂ fragment.

4. The isolated antibody or antigen binding fragment thereof of claim 1, wherein the antigen binding fragment is an scFv.

5. The isolated antibody or antigen binding fragment thereof of claim 1, wherein the antibody is an IgM antibody.

6. The isolated antibody or antigen binding fragment thereof of claim 1, wherein the ischemic antigen comprises SEQ ID NO: 17.

7. An isolated polypeptide comprising an amino acid sequence selected from the group consisting of SEQ ID NO: 8, SEQ ID NO: 10, SEQ ID NO: 12, and SEQ ID NO:16, which specifically binds to an ischemic antigen.

8. The isolated polypeptide of claim 7, wherein the ischemic antigen comprises SEQ ID NO: 17.

9. An isolated nucleic acid molecule selected from the group consisting of:
a) a nucleic acid molecule comprising a nucleotide sequence which is at least 98% identical to the nucleotide sequence of SEQ ID NO: 7, SEQ ID NO: 9, SEQ ID NO: 11, or SEQ ID NO:15; and
b) a nucleic acid molecule which hybridizes to the nucleotide sequence of SEQ ID NO: 7, SEQ ID NO: 9, SEQ ID NO: 11, or SEQ ID NO:15 under stringent conditions.

10. The isolated nucleic acid molecule of claim 9, which encodes a polypeptide comprising the amino acid sequence of SEQ ID NO: 8, SEQ ID NO: 10, SEQ ID NO: 12, or SEQ ID NO:16.

11. The isolated nucleic acid sequence of claim 9, which encodes an antibody or antigen binding fragment thereof which binds an ischemic antigen.

12. The isolated nucleic acid sequence of claim 11, wherein the ischemic antigen comprises SEQ ID NO: 17.

13. A vector comprising the nucleic acid of claim 9.

14. A cell comprising the vector of claim 13.


SEQUENCE LISTING (18 sequences)

SEQ ID NO: 1 - DNA, 402 bp, Mus musculus
caggttcagctgcagcagtctggggctgagctggtgaagcctggggcctcagtgaagatttcctgcaaagcttctggctacgcattcagtagctactggatgaactgggtgaagcagaggcctggaaagggtcttgagtggattggacagatttatcctggagatggtgatactaactacaacggaaagttcaagggcaaggccacactgactgcagacaaatcctccagcacagcctacatgcagctcagcagcctgacctctgaggactctgcggtctatttctgtgcaagagaagattactacggtagtgactggtacttcgatgtctggggcacagggaccacggtcaccgtctcctcaggtaagctggcttttttctttctgcacattccattctga

SEQ ID NO: 2 - protein, 133 aa, Mus musculus
QVQLQQSGAELVKPGASVKISCKASGYAFSSYWMNWVKQRPGKGLEWIGQIYPGDGDTNYNGKFKGKATLTADKSSSTAYMQLSSLTSEDSAVYFCAREDYYGSDWYFDVWGTGTTVTVSSGKLAFFFLHIPF

SEQ ID NO: 3 - DNA, 15 bp, Mus musculus
agctactggatgaac

SEQ ID NO: 4 - protein, 5 aa, Mus musculus
SYWMN

SEQ ID NO: 5 - DNA, 56 bp, Mus musculus
cagatttatcctggagatggtgatactaactacaacggaaagttcaagggcaaggc

SEQ ID NO: 6 - protein, 17 aa, Mus musculus
QIYPGDGDTNYNGKFKG

SEQ ID NO: 7 - DNA, 324 bp, Mus musculus
  CDS (1)..(324)
gatattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggctcggggacaaagttggaaataaaacgg

SEQ ID NO: 8 - protein, 108 aa, Mus musculus
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGSGTKLEIKR

SEQ ID NO: 9 - DNA, 33 bp, Mus musculus
  CDS (1)..(33)
aaggccagtcaggatgtgggtactgctgtagcc

SEQ ID NO: 10 - protein, 11 aa, Mus musculus
KASQDVGTAVA

SEQ ID NO: 11 - DNA, 21 bp, Mus musculus
  CDS (1)..(21)
tgggcatccacccggcacact

SEQ ID NO: 12 - protein, 7 aa, Mus musculus
WASTRHT

SEQ ID NO: 13 - DNA, 69 bp, Mus musculus
gaagattactacggtagtgactggtacttcgatgtctggggcacagggaccacggtcaccgtctcctca

SEQ ID NO: 14 - protein, 23 aa, Mus musculus
EDYYGSDWYFDVWGTGTTVTVSS

SEQ ID NO: 15 - DNA, 39 bp, Mus musculus
  CDS (1)..(39)
ctcacgttcggctcggggacaaagttggaaataaaacgg

SEQ ID NO: 16 - protein, 13 aa, Mus musculus
LTFGSGTKLEIKR

SEQ ID NO: 17 - protein, 12 aa, Mus musculus
LMKNMDPLNDNV

SEQ ID NO: 18 - protein, 15 aa, Artificial Sequence
  Description of Artificial Sequence Synthetic peptide
GGGGSGGGGSGGGGS